(19) United States
(12) Patent Application Publication
(10) Pub. No.: US 2021/0134399 A1
(43) Pub. Date: May 6, 2021

(54) CALCIUM ANALYSIS

(71) Applicants: Tampere University Foundation sr, TAMPEREEN YLIOPISTO (FI); TAYS Sydänkeskus Oy, TAMPERE (FI)

(72) Inventors: Katriina AALTO-SETÄLÄ, TAMPERE (FI); Kirsi PENTTINEN, NOKIA (FI); Martti JUHOLA, TAMPERE (FI); Henry JOUTSIJOKI, TAMPERE (FI)

(21) Appl. No.: 16/475,569

(22) PCT Filed: Dec. 28, 2017
(86) PCT No.: PCT/FI2017/050945

§ 371 Date: Jul. 2, 2019

(30) Foreign Application Priority Data
Jan. 2, 2017 (FI) 20175003

(51) Int. Cl. G16C 20/70; G01N 33/50; G01N 33/84; G06K 9/00

(52) U.S. Cl. CPC G16C 20/70; G01N 33/5061; G01N 2800/32; G06K 9/00147; G01N 33/84

(57) ABSTRACT

Disclosed is technique for calcium analysis on the basis of a calcium signal that includes a time series of samples that are descriptive of calcium level in a cardiomyocyte as a function of time is provided. According to an example, the technique involves a method that includes identifying calcium peaks in the calcium signal; Calculation of a calcium level, a calcium level, of a calcium level, of a calcium level, of a calcium level of at least one of at least one of at least one of at least temporal duration of the calcium peak, and a time difference to an adjacent calcium peak of the calcium signal. Also disclosed is a classifying method for determining the presence of different types of cells, and assigning the cardiomyocyte to one of the plurality of classes in accordance with the respective classifications.

### TECHNICAL FIELD

The example and non-limiting embodiments of the present invention relate to analysis of a signal that is descriptive of calcium level in a cardiomyocyte as a function of time.

### BACKGROUND

Patient specific, mutation specific or disease specific cardiomyocytes can be obtained with different cell technologies. Non-limiting examples of such technologies include differentiation of cardiomyocytes from reprogrammed stem cells, direct differentiation of cardiomyocytes and different genome altering techniques. Cardiomyocytes so obtained may be employed for study of cardiac functions of various types.

Calcium ions play a fundamental role in cardiac excitation-contraction coupling, which is crucial for a proper cardiomyocyte function and hence for a proper heart function. Depolarization and repolarization of a cardiomyocyte results in cyclically repeated increase and decrease in cytosolic calcium levels. These transient rises and reductions of cytosolic calcium control each cycle of contraction and relaxation of the heart. These calcium transients represent intracellular calcium levels in a cardiomyocyte. The calcium transients may be represented by a calcium signal that represents calcium level in a cardiomyocyte as a function of time.

Analysis of the calcium transients may be carried out, for example, in order to study cardiac functionality. Calcium cycling plays a major role in cardiac contractility and therefore alterations in calcium transients can be seen as contractile dysfunction and arrhythmogenesis associated with cardiac disorders and heart failures. Abnormalities in calcium transients may be seen e.g. as a variation in frequency and amplitude and they can be categorized by their form. By analyzing the calcium transients via inspection of a calcium signal that represents the calcium level in a cardiomyocytes, its cardiac functionality, possible cardiac disorders and drug responses can be studied more thoroughly.

A conventional technique for detection and analysis of abnormal calcium trans-sients involves visual inspection, detection and classification of abnormal transients in a calcium signal by a researcher. Since there are no generally accepted analysis criteria or tools for detection or classification of abnormal calcium transients, this conventional technique leads into subjective results. Moreover, such manual classification process is also relatively slow and repeatability of the process is typically poor.

In related art, WO 2015/158961 A1 discloses a technique for calcium level analysis on basis of a calcium signal that is descriptive of a calcium level in a cell as a function of time. The disclosed technique involves segmenting the calcium signal into a series of sections that each represent a respective peak in the calcium level and analyzing the change in calcium level within these sections in view of one or more detection rules to identify peaks that represent abnormal variations in the calcium level.

The following description further makes references to the following documents:
[1] L. Breiman, “Random Forests,” Machine Learning, Vol. 45, No. 1, pp. 5-32, 2001.
[2] H. Joutsijoki, M. Haponen, J. Rasku, K. Aalto-Setala and M. Juhola, “Machine learning approach to automated quality identification of human induced pluripotent stem cell colony images,” Computational and Mathematical Methods in Medicine, Vol. 2016(2016), Article ID 3091039, pp. 1-15, 2016.
[3] J. A. K. Suykens, T. van Gestel, J. De Brabanter, B. De Moor, J. Vandewalle, “Least squares support vector machines,” World Scientific, New Jersey, USA, 2002.
[4] J. A. K. Suykens and J. Vandewalle, “Least squares support vector machines,” Neural Processing Letters, Vol. 9, No. 3, pp. 293-300, 1999.
[5] J. A. K. Suykens and J. Vandewalle, “Multiclass least squares support vector machines,” Proceedings of the International Joint Conference on Neural Networks, Vol. 2, pp. 900-903, 1999.
[6] S. A. Dudani, “The Distance-Weighted k-Nearest-Neighbor Rule,” IEEE Transactions on Systems, Man, and Cybernetics, vol. 6, no. 4, pp. 325-327, 1976.
[7] T. Cover and P. Hart, “Nearest neighbor pattern classification,” IEEE Transactions on Information Theory, vol. 13, no. 1, pp. 21-27, 1967.

### SUMMARY

It is an object of the present invention to provide an analysis technique that enables estimation of a cardiac condition via analysis of a calcium signal in a reliable, objective and repeatable manner.

According to an example embodiment, a method for calcium analysis on basis of a calcium signal that comprises a time series of samples that are descriptive of calcium level in a cardiomyocyte as a function of time is provided, the method comprising identifying calcium peaks in the calcium signal; deriving, for each identified calcium peak, respective values for a plurality of peak characteristics that include at least one of the following: a change in calcium level indicated by the calcium peak, a rate of change in calcium level indicated by the calcium peak, a temporal duration of the calcium peak, and a time difference to an adjacent calcium peak of the calcium signal; classifying each identified calcium peak into one of a plurality of classes on basis of said values derived for the respective peak in dependence of predefined classification information that represents said plurality of classes, wherein each of said plurality of classes represents a respective predetermined cardiac condition; and assigning said cardiomyocyte to one of said plurality of classes in accordance with the respective classifications.

According to another example embodiment, an apparatus for calcium analysis on basis of a calcium signal that comprises a time series of samples that are descriptive of a calcium level in a cardiomyocyte as a function of time is provided, the apparatus comprising means for identifying calcium peaks in the calcium signal; means for deriving, for each identified calcium peak, respective values for a plurality of peak characteristics that include at least one of the following: a change in calcium level indicated by the calcium peak, a rate of change in calcium level indicated by the calcium peak, a temporal duration of the calcium peak, and a time difference to an adjacent calcium peak of the calcium signal; means for classifying each identified calcium peak into one of a plurality of classes on basis of said values derived for the respective peak in dependence of predefined classification information that represents said plurality of classes, wherein each of said plurality of classes represents a respective predetermined cardiac condition; and means for assigning said cardiomyocyte to one of said plurality of classes in accordance with the respective classifications.

According to another example embodiment, an apparatus for calcium analysis on basis of a calcium signal that comprises a time series of samples that are descriptive of a calcium level in a cardiomyocyte as a function of time is provided, the apparatus comprising at least one processor and at least one memory including computer program code for one or more programs, the at least one memory and the computer program code configured to, with the at least one processor, cause the apparatus at least to: identify calcium peaks in the calcium signal; derive, for each identified calcium peak, respective values for a plurality of peak characteristics that include at least one of the following: a change in calcium level indicated by the calcium peak, a rate of change in calcium level indicated by the calcium peak, a temporal duration of the calcium peak, and a time difference to an adjacent calcium peak of the calcium signal; classify each identified calcium peak into one of a plurality of classes on basis of said values derived for the respective peak in dependence of predefined classification information that represents said plurality of classes, wherein each of said plurality of classes represents a respective predetermined cardiac condition; and assign said cardiomyocyte to one of said plurality of classes in accordance with the respective classifications.

According to another example embodiment, a computer program for calcium level analysis on basis of a calcium signal that is descriptive of a calcium level in a cardiomyocyte as a function of time is provided, the computer program including one or more sequences of one or more instructions which, when executed by one or more processors, cause an apparatus at least to carry out the method according the example embodiment described in the foregoing.

The computer program referred to above may be embodied on a volatile or a non-volatile computer-readable record medium, for example as a computer program product comprising at least one computer readable non-transitory medium having program code stored thereon, the program which when executed by a computing apparatus causes the apparatus at least to carry out the method according the example embodiment described in the foregoing.

The exemplifying embodiments of the invention presented in this patent application are not to be interpreted to pose limitations to the applicability of the appended claims. The verb “to comprise” and its derivatives are used in this patent application as an open limitation that does not exclude the existence of also unrecited features. The features described hereinafter are mutually freely combinable unless explicitly stated otherwise.

Some features of the invention are set forth in the appended claims. Aspects of the invention, however, both as to its construction and its method of operation, together with additional objects and advantages thereof, will be best understood from the following description of some example embodiments when read in connection with the accompanying drawings.

### BRIEF DESCRIPTION OF FIGURES

The embodiments of the invention are illustrated by way of example, and not by way of limitation, in the figures of the accompanying drawings where

FIG. 1 schematically illustrates an extract of an exemplifying calcium signal;

FIGS. 2A and 2B depict respective calcium signals obtained from a patient suffering from long QT syndrome 1 (LQT1);

FIGS. 2C and 2D depict respective calcium signals obtained from a patient suffering from hypertrophic cardiac myopathy (HCM);

FIGS. 2E and 2F depict respective calcium signals obtained from a patient suffering from catecholaminergic polymorphic ventricular tachycardia (CPVT);

FIG. 3 depicts a flowchart illustrating a method according to an example embodiment;

FIG. 4 schematically depicts a calcium peak, its first derivative and its second derivative;

FIG. 5 illustrates a block diagram depicting some elements of a calcium analyzer according to an example embodiment; and

FIG. 6 illustrates a block diagram depicting some elements of a computing device according to an example embodiment.

### DESCRIPTION OF SOME EMBODIMENTS

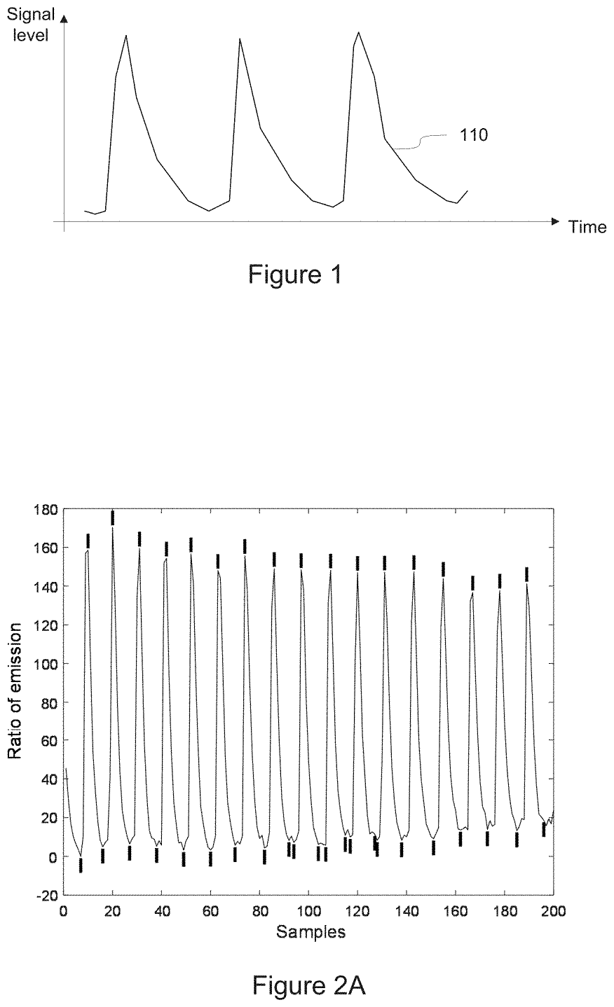

FIG. 1 schematically illustrates an extract of an exemplifying calcium signal 110. As described in the foregoing, the calcium signal 110 represents the calcium level (y axis of the graph) as a function of time (x axis of the graph). In the following, for editorial clarity of the description, the difference in calcium level may be referred to as a distance in direction of the y axis or as a vertical distance. Along similar lines, the difference in time may be referred to as a distance in direction of the x axis or as a horizontal distance. As used herein, however, the terms ‘vertical’ and ‘horizontal’ have no spatial significance but rather refer to illustration of the calcium level in the figures of the present application.

As described in the foregoing, the calcium level may serve as an indication of the intracellular calcium level in a cardiomyocyte. The calcium signal 110 is a digital signal comprising a sequence of samples, each representing measured calcium level at a respective time instant. The samples constituting the calcium signal 110 preferably exhibit regular spacing in time, in other words the (digital) calcium signal 110 represents the calcium level at a constant sample rate (i.e. sample frequency). As a typical but non-limiting example, the sample rate may be in the range from 10 to 100 Hz, e.g. 20 Hz and, consequently, the samples of the calcium signal 110 may represent the calcium level at 10 to 100 millisecond intervals, e.g. at 50 millisecond intervals. In other examples, sample rates outside of this range may be employed instead.

The calcium signal 110 may be obtained using any suitable technique known in the art. As an example in this regard, the calcium signal 110 may be obtained on basis of calcium imaging. In this regard, the calcium imaging may be conducted for one or more spontaneously beating, calcium indicator loaded dissociated cardiomyocytes that are perfused with extracellular solution, wherein the calcium indicators comprises fluorescent indicators. Calcium imaging may be based on exciting the fluorescent indicators with a light of suitable wavelength(s) and recording the level or intensity of light emitted by the fluorescent indicators (at a certain wavelength) in response to the excitation. Hence, the recorded information comprises a time series of light level/intensity values, thereby serving as basis for deriving the calcium signal 110 representing the calcium level as a function of time. The calcium signal 110 may be formed e.g. as the recorded light level/intensity obtained on basis of excitation using a single excitation wavelength or as the ratio of recorded light levels/intensities obtained on basis of excitation using two different excitation wavelengths.

The calcium imaging may be based on calcium measurements that are carried out by using e.g. an inverted microscope equipped with an objective that is suitable for capturing light emitted by the fluorescent markers. Moreover, a suitable light source for generating the light serving as excitation for the fluorescent indicator and a suitable imaging device (e.g. a digital camera) for capturing images representing the light emitted by the fluorescent markers may be employed, together with control logic (e.g. a digital signal processor (DSP) with an appropriate software) for operating the light source and the imaging device, for extracting the emitted light levels/intensities on basis of the captured image and for recording (in a memory) the extracted light levels/intensities as a function of time.

As an example of an arrangement for obtaining the calcium signal 110 via calcium imaging as outlined in the foregoing, e.g. the IX70 inverted microscope (by Olympus Corporation, Hamburg, Germany) together with the UApo/340 x20 air objective (by Olympus Corporation) may be employed. Moreover, the images may be capture using e.g. the ANDOR iXon 885 CCD camera (by Andor Technology, Belfast, Northern Ireland) synchronized e.g. with a Polychrome V light source by a real time DSP control unit and TILLvisION or Live Acquisition software (by TILL Photonics, Munich, Germany). The calcium indicators may be excited using light at 340 nanometer (nm) and/or 380 nm wavelength and the emissions are may be recorded at 505 nm wavelength. For further calcium analysis, regions of interest may be selected for spontaneously beating (dissociated) cardiomyocytes and the calcium signal 110 may be acquired as the level/intensity of light emitted in response to the excitation signal at 340 nm or 380 nm wavelengths or as the ratio of the levels/intensities of light emitted in response to excitation signals at 340 nm and 380 nm wavelengths.

The resulting calcium signal 110 may be transferred to an analysis tool or to an analysis device for further analysis for example as a data file (e.g. as a text file) comprising indications of the time instants and corresponding fluorescence intensities or fluorescence intensity ratios. Alternatively, the data file may only include indications of the fluorescence intensities or ratios that represent the fluorescence intensity or ratio at a predefined sample rate. The analysis device/tool may be e.g. an analyzer device that is provided with means for implementing an analysis method described in detail via various examples in the following.

Typically, the calcium level exhibits cyclic variation over time, which results in a time series of transients in the calcium level, represented by a corresponding time series of transients of the calcium signal 110. A calcium transient may also be referred to as a calcium peak. In this text, the terms calcium transient and calcium peak are used interchangeably. As an example, the extract of the calcium signal 110 illustrated in FIG. 1 depicts a sequence of three calcium peaks.

Normal variation in calcium level that typically does not suggest an unhealthy condition involves constant or substantially constant pattern of change in the calcium level that repeats at constant or substantially constant rate. In the calcium signal 110 such normal variation is represented by a sequence of normal peaks that exhibit constant or substantially constant height and overall shape at regular or substantially regular (temporal) spacing between consecutive peaks of the sequence. However, even for a healthy subject the height, the shape and/or the (temporal) spacing of the peaks of the calcium signal 110 may exhibit small variations and/or gradual evolution over time.

In contrast, in case the calcium signal 110 that fails to indicate constant or substantially constant pattern of change in the calcium level and/or where the pattern of change fails to repeat at constant or substantially constant rate may in some cases serve as an indication of an unhealthy cardiac condition. In this regard, a calcium signal that represents or is likely to represent an unhealthy cardiac condition includes one or more abnormal peaks that substantially differ from a normal peak e.g. in height, in shape and/or in (temporal) spacing to one an adjacent peak, and such a calcium signal is detectable via analysis of peak structure of the calcium signal 110. Moreover, even constant or substantially constant pattern of change in the calcium level that repeats at constant or substantially constant rate may hide an unhealthy cardiac condition that may also be detectable via computational analysis of the peak structure of the calcium signal 110.

As a particular example, analysis of the calcium signal 110 may be carried out in order to determine whether it represents one of a plurality of (i.e. two or more) predetermined cardiac conditions. In this regard, two basic scenarios can be identified:
The calcium signal analysis may be carried out in order to determine which one of the (two or more) predetermined unhealthy cardiac conditions is most likely in view of the calcium signal 110. In other words, the predetermined cardiac conditions considered in the calcium signal analysis may comprise two or more predetermined unhealthy cardiac conditions of different characteristics.
The calcium signal analysis may be carried out in order to determine whether a healthy cardiac condition or one of one or more predetermined unhealthy cardiac conditions is most likely in view of the calcium signal 110. In other words, the predetermined cardiac conditions considered in the calcium signal analysis may comprise a healthy cardiac condition together with one predetermined unhealthy cardiac condition or with two or more predetermined unhealthy cardiac conditions of different characteristics.

The one or more unhealthy cardiac conditions considered in the calcium signal analysis may comprise one or more inheritable cardiac conditions, e.g. one or more of the following: catecholaminergic polymorphic ventricular tachycardia (CPVT), which is an exercise-induced malignant arrhythmogenic disorder, long QT syndrome 1 (LQT1), which is an electric disorder of the heart that predisposes patients to arrhythmias and sudden cardiac death, and hypertrophic cardiac myopathy (HCM), a disorder that affects the structure of heart muscle tissue leading to arrhythmias and progressive heart failure. However, CPVT, LQT1 and HCM serve as non-limiting examples of unhealthy cardiac conditions that may be considered in the calcium signal analysis.

An outcome of the calcium signal analysis may comprise, for example, an indication of the most likely one of the cardiac conditions under consideration. In another example, the outcome of the calcium signal analysis comprises respective indications of relative probabilities for the cardiac conditions under consideration. In both these scenarios the outcome of the calcium signal analysis may be provided for further analysis and/or for use as basis for a diagnosis by a medical practitioner.

Before and/or in the course of analysis, the calcium signal 110 under analysis may be pre-classified either as a normal signal or an abnormal signal and an indication of the outcome of the pre-classification may be provided as input for the calcium analysis. In an example, the indication whether the calcium signal 110 under analysis is considered as a normal signal or an abnormal signal may be used in the calcium analysis procedure to select and/or adjust classification of the calcium signal 110 to likely represent one of a plurality of predetermined cardiac conditions.

In an example, such pre-classification may be carried out by a human observer (e.g. a medical practitioner). Such pre-classification may be based e.g. on visual analysis of one or more curves that represent the calcium signal 110 under analysis. In another example, the pre-classification may be carried out by using a computational analysis of the calcium signal 110 under consideration. An example of such pre-classification is described in detail in WO 2015/158961 A1. In a further example, the pre-classification may be a combination of an automated classification by computational analysis and visual inspection by a human observer, e.g. such that the automated classification is verified and corrected where needed via visual inspection by a human expert.

Regardless of the manner of carrying out the pre-classification procedure (e.g. by a human observer, by a computational technique, by a combination of the two), the applied pre-classification criteria may be selected according to desired sensitivity of pre-classifying the calcium signal 110 under consideration as an abnormal one. In an example, the calcium signal 110 may be pre-classified as an abnormal signal in response to identifying at least one abnormal calcium peak therein and pre-classified as a normal signal in response to absence of abnormal peaks. In another example, the calcium signal 110 may be pre-classified as an abnormal signal in response to identifying at least a predefined amount of abnormal peaks therein and pre-classifying the calcium signal 110 as a normal signal otherwise. In the latter scenario, the predefined amount may be defined via an absolute number of abnormal peaks, via a percentage of abnormal peaks, or via combination of the two.

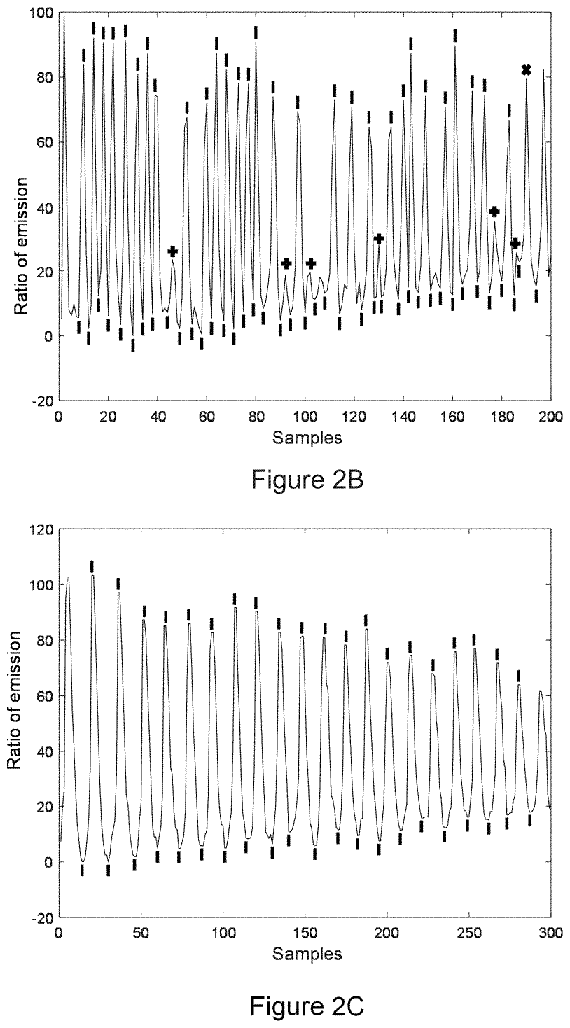

As further background concerning normal and abnormal calcium signals, FIGS. 2A to 2F schematically illustrate non-limiting examples in this regard. Throughout these illustrations, the depicted calcium signal is pre-classified as a normal or abnormal one by using an automated calcium peak detection technique to identify abnormal peaks in a calcium signal, followed by classification of the calcium signal as normal or abnormal one via visual inspection by a human expert.

FIG. 2A depicts a calcium signal obtained from cardiomyocytes derived from reprogrammed stem cells that are obtained from a patient carrying a gene mutation for long QT syndrome (LQTS) and having symptoms of LQT1. In this example, all calcium peaks that appear in the illustration in their entirety have been pre-classified as normal ones (marked with a rectangular marker on top) by an automated calcium peak detection and classification technique, whereas the depicted calcium signal has been pre-classified as a normal signal by the human expert.

FIG. 2B depicts a calcium signal from cardiomyocytes obtained from a patient suffering from LQT1. In this example, the automated calcium peak detection and classification technique has resulted in pre-classifying most calcium peaks that appear in the illustration in their entirety as normal ones (marked with a rectangular marker on top), whereas eight calcium peaks have been pre-classified as abnormal ones (marked with a plus sign or a cross-shaped marker on top) and the depicted calcium signal has been pre-classified as an abnormal signal by the human expert.

FIG. 2C depicts a calcium signal obtained from cardiomyocytes derived from stem cells that are obtained from a patient carrying a mutation for HCM and having clinical findings of HCM. In this example, all calcium peaks that appear in the illustration in their entirety have been pre-classified as normal ones (marked with a rectangular marker on top) by the automated calcium peak detection and classification technique and the depicted calcium signal has been hence pre-classified as normal signal by the human expert.

FIG. 2D depicts a calcium signal from cardiomyocytes obtained from a patient suffering from HCM. In this example, the automated calcium peak detection and classification technique has resulted in pre-classifying most calcium peaks that appear in the illustration in their entirety as normal ones (marked with a rectangular marker on top), whereas four calcium peaks have been pre-classified as abnormal ones (marked with a cross-shaped marker on top) and, consequently, the depicted calcium signal has been pre-classified as an abnormal signal by the human expert.

FIG. 2E depicts a calcium signal obtained from cardiomyocytes derived from reprogrammed stems cells obtained from a patient having mutation in RyR2 gene and suffering from symptoms of CPVT. In this example, all calcium peaks that appear in the illustration in their entirety have been pre-classified as normal ones (marked with a rectangular marker on top) by the automated calcium peak detection and classification technique and the depicted calcium signal has been hence pre-classified as a normal signal by the human expert.

FIG. 2F depicts a calcium signal from cardiomyocytes obtained from a patient suffering from CPVT. Considering the calcium peaks that appear in their entirety in this example, the automated calcium peak detection and classification technique has resulted in pre-classifying a half of the calcium peaks as normal ones (marked with a rectangular marker on top), whereas the other half have been pre-classified as abnormal ones (marked with a cross-shaped marker on top). Consequently, the depicted calcium signal has been pre-classified as an abnormal signal by the human expert.

Considering the pre-classification of the calcium signal in view of the examples of FIGS. 2A to 2F and assuming that even a single abnormal calcium peak is sufficient to render the calcium signal as an abnormal one, the example signals of FIGS. 2B, 2D and 2F represent abnormal calcium signals, whereas the example signals of FIGS. 2A, 2C and 2E represent normal calcium signals. However, pre-classification of the calcium signal as a normal signal does not, as such, indicate that the signal represents a healthy cardiac condition or pre-classification as an abnormal signal does not indicate that the signal represents an unhealthy cardiac condition: a calcium signal may represent either a healthy or unhealthy cardiac condition regardless of its pre-classification as a normal or abnormal signal, while in some scenarios the pre-classification may serve to enable improved calcium analysis.

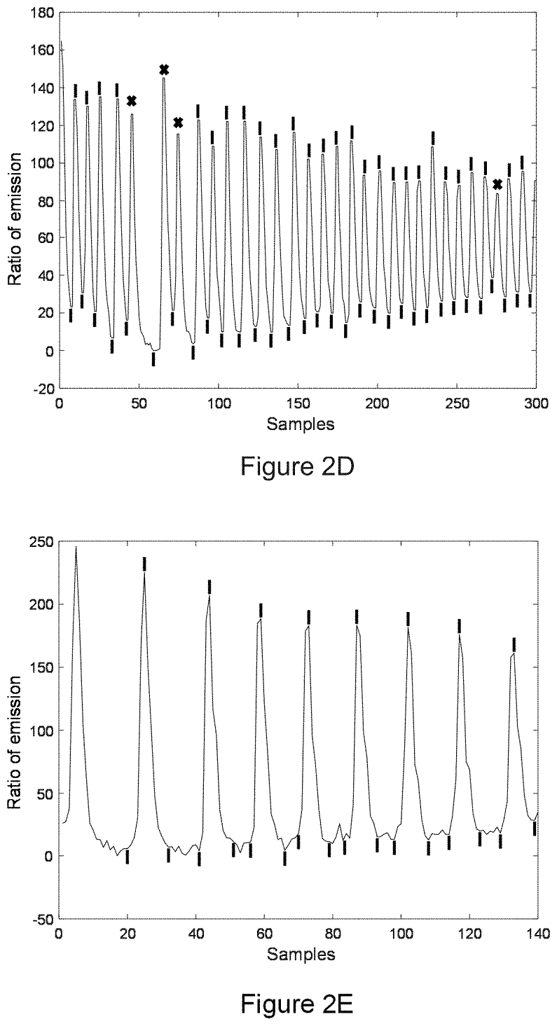

FIG. 3 depicts a flowchart that illustrates steps of a method 300 for calcium analysis according to various examples outlined in the following. The method 300 commences from obtaining the calcium signal 110, as indicated in block 310. In an example, the procedure of block 310 comprises obtaining the calcium signal 110 by carrying out calcium imaging according to the procedure outlined in the foregoing. In another example, the procedure of block 310 comprises reading the calcium signal 110 from a memory device connected to or provided in a computing apparatus that is arranged to implement the method 300 or receiving the calcium signal 110 via a communication interface. In context of method 300, the calcium signal 110 is provided as a time series of samples or sample values that each represent the calcium level at respective time instant. In other words, the samples of the calcium signal 110 are descriptive of the calcium level (in a cardiomyocyte) as a function of time.

In an example, the method 300 may commence from a priori knowledge that the calcium signal 110 under analysis originates from a cardiomyocyte obtained from a person that suffers from an inheritable cardiac condition and the calcium analysis according to the method 300 serves to determine respective relative probabilities of two or more predetermined unhealthy cardiac conditions. In another example, there is no a priori knowledge of the health of the person from whom the cardiomyocyte whose calcium level is represented by the calcium signal 110 under analysis, and the calcium analysis according to the method 300 serves to determine respective relative probabilities of a healthy cardiac condition and one or more predetermined unhealthy cardiac conditions.

The method 300 comprises identifying calcium peaks in the calcium signal 110 under analysis, as indicated in block 320. Conceptually, a calcium peak in the calcium signal 110 is defined by a pair local minima in the calcium signal 110 and the local maxima between these local minima: the local minimum of the pair that appears first in the calcium signal 110 represents the beginning of a peak, the local maxima represents the top of the peak, and the local minimum of the pair that appears later in the calcium signal 110 represents the end of the peak. Consequently, samples of the calcium signal 110 that represent a signal segment from the beginning of the peak to the top of the peak constitute an ascending side (left side) of the peak, whereas samples of the calcium signal 110 that represent a signal segment from the top of the peak to the end of the peak constitute a descending side (right side) of the peak.

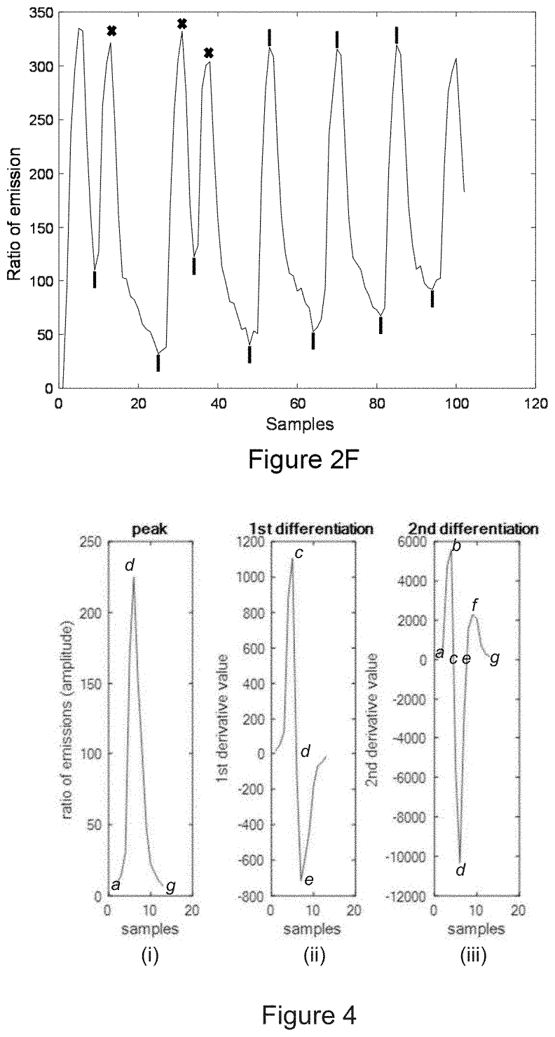

As an example in this regard, the illustration (i) in FIG. 4 schematically depicts a calcium peak, where sample position a denotes the beginning of the peak, point d denotes the top of the peak and sample position g denotes the end of the peak. Moreover, the ascending side of the depicted peak runs from sample position a to sample position d, whereas the descending side runs from sample position d to sample position g.

In an example, identification of the calcium peaks in the calcium signal 110 involve using an automated peak detection, for example the one described in WO 2015/158961 A1. This, however, serves as a non-limiting example of an automated calcium peak detection procedure and another calcium peak detection procedure known in the art may be applied instead. Alternatively, the calcium peak identification may be carried out as a manual procedure by a human expert via visual inspection of a curve that represents the calcium signal 110. In a further example, a combination of the automated peak detection and manual procedure may be employed, e.g. such that outcome of the automated peak detection procedure is followed by a visual inspection by a human expert to verify the outcome of the automated procedure and to correct and/or complement the outcome of the automated procedure where needed.

Regardless of the manner of carrying out the procedure (automated, manual, a combination of the two), the outcome the peak identification comprises information that defines the calcium peaks in the calcium signal 110, e.g. by defining, for each identified peak, the beginning of the peak (e.g. sample position a), the top of the peak (e.g. sample position d) and the end of the peak (e.g. sample position g).

After identification of the calcium peaks in the calcium signal 110, the method 300 proceeds to derivation of respective values of a plurality of peak characteristics for each identified peak of the calcium signal 110, as indicated in block 330. The considered peak characteristics may be descriptive of one or more of the following: a change in calcium level indicated by the peak, a rate of change in calcium level indicated by the peak, a temporal duration of the peak, temporal spacing to an adjacent peak. In a particular example, one or more of the following peak characteristics may be considered:
#1. Change in calcium level indicated by the ascending side of a peak, represented in the calcium signal 110 by the amplitude of the peak from the beginning of the peak to the top of the peak;
#2. Change in calcium level indicated by the descending side of a peak, represented in the calcium signal 110 by the amplitude of the peak from the top of the peak to the end of the peak;
#3. Duration of the ascending side of the peak, represented in the calcium signal 110 by time difference between the beginning of the peak and the top of the peak;
#4. Duration of the descending side of the peak, represented in the calcium signal 110 by time difference between the top of the peak and the end of the peak;
#5. Maximum rate of change in calcium level in the ascending side of a peak, represented by the maximum of the first derivative of the calcium signal 110 in a segment from the beginning of the peak to the top of the peak;
#6. Absolute value of minimum rate of change in calcium level in the descending side of a peak, represented by the absolute value of the minimum of the first derivative of the calcium signal 110 in a segment from the top of the peak to the end of the peak;
#7. Maximum change in the rate of change in calcium level in the descending side of a peak, represented by the maximum of the second derivative of the calcium signal 110 in a segment from the top of the peak to the end of the peak;
#8. Absolute value of minimum change in the rate of change in calcium level in the descending side of a peak, indicated by the absolute value of the minimum of the second derivative of the calcium signal 110 in a segment from the top of the peak to the end of the peak;
#9. An area defined by a peak, indicated by the are limited by a segment of the calcium signal 110 from the beginning of the peak to the end of the peak together with a(n imaginary) line connecting the beginning of the peak to the end of the peak;
#10. Time difference between a peak and the immediately preceding peak, represented by the time difference between the top of the peak and the top of the immediately preceding peak of the calcium signal 110. Alternatively, the time difference may be indicated between the top of the peak and the top of the immediately following peak of the calcium signal.

The exemplifying peak characteristics #1 to #10 introduced in the foregoing are provided in no particular order and the numbering applied therefor merely serves as identification of the listed peak characteristics to enable conveniently referring back to some of these peak characteristics in the following.

To further illustrate some of the peak characteristics described in the foregoing, the illustration (ii) of FIG. 4 schematically illustrates the first derivative of the calcium signal representing the calcium peak of the illustration (i). Therein, sample position c denotes the point of maximum rate of change in the calcium level in the ascending side of the peak (e.g. the peak characteristic #5), and sample position e denotes the point of minimum rate of change in the calcium level in the descending side of the peak (e.g. the peak characteristic #6). The illustration (iii) of FIG. 4 schematically illustrates the second derivative of the calcium signal representing the calcium peak of the illustration (i). Therein, sample position f denotes the point of maximum change in the rate of change in the calcium level in the ascending side of the peak (e.g. the peak characteristic #7), and sample position d denotes the minimum change in the rate of change in the calcium level in the descending side of a peak (e.g. the peak characteristic #8).

After derivation of the peak characteristic values (cf. block 330) for the identified peaks of the calcium signal 110, the method 300 proceeds to classification of the identified peaks into one of a plurality of classes on basis of the peak characteristic values derived for the respective peak, as indicated in block 350. Hence, each calcium peak is separately classified into one of the classes on basis of the peak characteristic values derived therefor. Each of the classes represents a respective predetermined cardiac condition and the classification of the identified calcium peaks to these classes is carried out in dependence of predefined classification information that defines mapping between the peak characteristics and the class. The classification information will be described in more detail in the following.

The classification of block 350 is followed by assignment of the cardiomyocyte that the calcium signal 110 under analysis represents into one or more of the above-mentioned classes in accordance with the classifications, as indicted in block 360. In an example, the assignment involves assigning the cardiomyocyte to represent the class that has the highest number of calcium peaks classified thereto via operation of block 350. In a scenario where there are two or more classes that have the (equal) highest number of calcium peaks classified thereto one of the following approaches may be applied:
The cardiomyocyte represented by the calcium signal 110 under analysis is assigned to represent all of the two or more classes that have the highest number of calcium peaks classified thereto.
The cardiomyocyte represented by the calcium signal 110 under analysis is assigned to represent randomly selected one of the two or more classes that have the highest number of calcium peaks classified thereto.
The cardiomyocyte represented by the calcium signal 110 under analysis is assigned to represent one of the two or more classes that have the highest number of calcium peaks classified thereto via application of a tie-breaking rule. An example of a tie-breaking rule is described later in this text.

The outcome of the assignment (of block 360) may be further output for further analysis and/or for use as basis for a diagnosis by a medical practitioner, as indicated in block 370. In an example, the outcome of the assignment includes an indication of the class to which the cardiomyocyte under analysis (via analysis of the calcium signal measured therefrom) is assigned, which may be considered as an indication of the most likely one of the cardiac conditions under consideration via operation of the method 300. In another example, the outcome of the assignment includes, additionally or alternatively, a respective indication or estimate of relative probability of one or more of the classes considered in the classification procedure of block 360. In this regard, the outcome of the assignment may comprise a respective indication of the ratio between the number of calcium peaks classified into a certain class divided by the overall number of considered calcium peaks e.g. for one or more classes having the highest number of calcium peaks classified thereto or for all classes considered in the classification procedure.

The classification information applied in the classification procedure of block 350 serves to define a mapping between a set of peak characteristic values derived for a calcium peak and the considered classes. The classification information may also be referred to as classification data. In order to enable the mapping, the classification information comprises predefined mapping information derived on basis of training data via a training procedure. The training procedure is typically carried out before operation of the method 300, thereby making the classification information readily available for computationally efficient calcium analysis. Alternatively, the training procedure may be carried out in the course of the method 300 (e.g. as part of the method 300) before proceeding to the assignment of the identified peaks to available classes in block 350. The latter approach is computationally more demanding and also requires access to the training data during operation of the method 300. On the other hand, it enables continuously complementing the training data with new information derived from the calcium peaks under analysis by the method 300, thereby facilitating improved accuracy of the classification procedure via repeated operations of the method 300.

The training data includes a respective sub-set of data for each cardiac condition to be considered in the analysis according to the method 300. Each sub-set includes respective set of peak characteristic values derived for a plurality of calcium peaks that are known to represent the respective cardiac condition. The peak characteristics included in the training data are the same ones that are derived for calcium peaks identified in the calcium signal 110 (in block 330) and that are considered in the classification procedure (in block 350) in the course of the method 300.

If assuming a classification/training procedure that employs K different peak characteristic, the training procedure results in mapping information that, depending on the applied training/assignment approach, defines a respective reference point or a respective partition in a K-dimensional space for each of the considered classes. Furthermore, without losing generality, a set of peak characteristic values derived for a calcium peak (e.g. in block 330) may be (at least conceptually) arranged as a K-dimensional peak vector, where each peak characteristic value is provided as one element of the peak vector, the peak vector thereby defining a point in the K-dimensional space. In this regard, the classification procedure (e.g. block 350) may involve assigning a calcium peak via the point in the K-dimensional space that represents the calcium peak e.g. in one of the following ways (depending on the employed training/assignment approach):
assign a calcium peak into class whose reference point is closest to the point that represents the calcium peak in the K-dimensional space in view of a predefined distance measure;
assign a calcium peak into class whose partition of the K-dimensional space includes the point that represents the calcium peak in the K-dimensional space.

The description of the foregoing briefly outlines examples of classification approaches together with the underlying training procedure that are applicable in context of the method 300. In general, a multitude of applicable training and classification approaches are available in the art and the choice of the most advantageous one depends on the number and characteristics of the underlying cardiac conditions under consideration via the calcium analysis according to the method 300. However, in particular the following classification approaches are found to yield reliable analysis via operation of the method 300 when carrying out the calcium analysis:
a random forests classifier, described in detail for example in [1];
a binary tree least-squares support vector machine (BT-LSSVM) classifier with radial basis function kernel (RBF), described in detail for example in [2] and the basic theory of a two-class LSSVM can be found e.g. from [3], [4] and [5];
a K-nearest neighbor method (k-NN) [6] and [7].

The tie-breaking rule referred to in the foregoing in context of block 360 may rely on the training data in order to select one of the two or more classes that have the (equal) highest number of calcium peaks classified thereto via operation of block 350. As a non-limiting example in this regard, the tie-breaking rule may involve the following steps:
1. Assume two classes C₁ and C₂ that both have the (equal) highest number of calcium peaks assigned thereto;
2. Identify the number of calcium peaks in the training data for the two classes C₁ and C₂ and denote these numbers of calcium peaks by P₁ and P₂, respectively;
3. Generate a random number R using a uniform distribution U(0,1); and
4. If R<P₁/(P₁+P₂), then the assign the cardiomyocyte into class C₁, otherwise assign the cardiomyocyte into class C₂.

The method 300 may optionally further comprise pre-classifying the calcium signal 110 as one of a normal signal or an abnormal signal, as indicated in block. 340. Such pre-classification has been introduced and described via a number of examples in the foregoing. As described therein, pre-classification of the calcium signal 110 as a normal signal does not as such imply a healthy cardiac condition or pre-classification of the calcium signal 110 as an abnormal signal does not imply an unhealthy cardiac condition, but the outcome of the pre-classification may be used in the method 300 to select and/or adjust classification of calcium peaks identified in the calcium signal 110.

As an example in this regard, the method 300 may have access to two different sets of classification data, where a first set is prepared for classification of calcium signals pre-classified as normal signals, while a second set is prepared for classification of calcium signals pre-classified as abnormal signals. Consequently, the pre-classification of block 340 results in selecting the first set of classification data in response to finding the calcium signal 110 to represent a normal signal and selecting the second set of classification data in response to finding the calcium signal 110 to represent an abnormal signal. In this regard, the first set of classification data may be prepared, e.g. according to the training procedure outlined in the foregoing, using training data that comprises peak characteristic values extracted from calcium signals and/or calcium peaks that represent normal calcium signals, whereas the second set of data may be prepared using training data that comprises peak characteristic values extracted from calcium signals and/or calcium peaks that represent abnormal calcium signals. Preferably, the same or similar criteria as in pre-classification of block 340 is applied also in selection of the calcium signals and/or calcium peaks for use as the training data in order to ensure properly accounting for possible differences in peak characteristic values between calcium signals considered as normal and abnormal.

In another example, the method 300 may employ a single set of classification data but e.g. the reference points or the partitions that at least in part define the classification within the classification data are adjusted in accordance with the outcome of the pre-classification. As an example in this regard, reference points in the classification data may be defined for a calcium signal that is pre-classified as a normal signal and used as such in response to finding the calcium signal 110 to represent a normal signal, whereas the reference points may be adjusted in a predefined manner for use for a calcium signal that is pre-classified as an abnormal signal and the adjusted reference points are applied instead in response to finding the calcium signal 110 to represent an abnormal signal.

Several experiments have been carried out to validate the approach described in the foregoing via a number of examples. These experiments involve, among others, the following:
an experiment where the predetermined cardiac conditions consisted of CPVT, LQT1 and HCM; and
an experiment where the predetermined cardiac conditions consisted of CPVT, LQT1 and HCM together with a healthy cardiac condition,
   where both of the above sets of predetermined cardiac conditions where repeated using the following pre-classification approaches:
a classification of calcium signals that are pre-classified as normal signals on basis of classification data derived from calcium signals that are pre-classified as normal signals;
a classification of calcium signals that are pre-classified as abnormal signals on basis of classification data derived from calcium signals that are pre-classified as abnormal signals; and
a classification of non-pre-classified calcium signals on basis of classification data derived from non-pre-classified calcium signals.

Moreover, the experiments outlined in the foregoing we repeated using several different classification techniques known in the art, including the random forests classifier, the BT-LSSVM classifier with a RBF and the k-NN classifier together with a number of other classification techniques known in the art, where the classification techniques identified herein were found to yield the best classification performance.

Throughout these experiments the results indicate that good classification performance can be obtained by using a single peak characteristic. As an example in this regard, the classification (e.g. via the method 300) may consider only a single peak characteristic considered in the classification, where the single peak characteristic comprises one of the exemplifying peak characteristics #3, #4 or #10.

On the other hand, the experimental results further indicate that classification performance can be improved by using two or three peak characteristics. Hence, in another example two or three peak characteristics are considered in the classification, where the considered peak characteristics comprise one of the following combinations:
the exemplifying peak characteristics #3, #4 and #10;
the exemplifying peak characteristics #3 and #4;
the exemplifying peak characteristics #3 and #10;
the exemplifying peak characteristics #4 and #10;

The experimental results further indicate that at least some further improvement in the classification performance can be obtained by using more than three peak characteristic. Hence, in a further example, the peak characteristics #1 to #10 are considered in the classification.

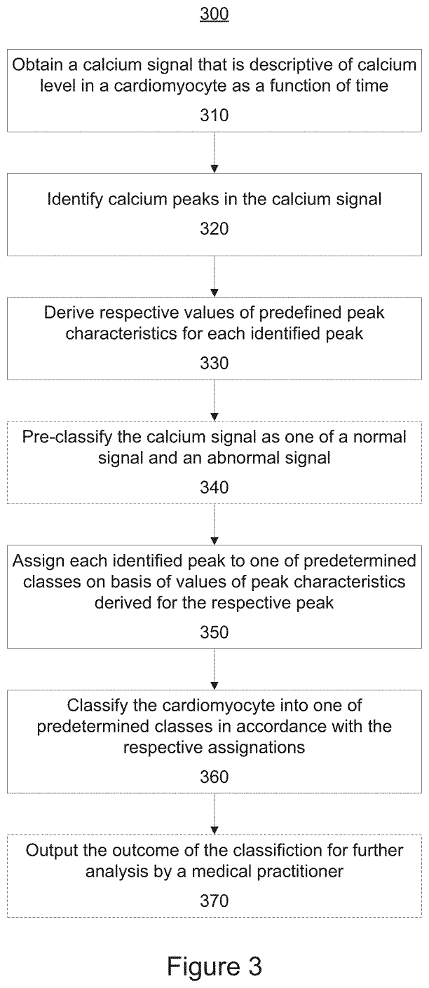

The method 300 may be implemented by an apparatus or device, and such apparatus/device may be referred to as calcium analyzer. FIG. 5 illustrates a block diagram that depicts some elements of a calcium analyzer 500 according to an example. The input to the calcium analyzer 500 is the calcium signal 110. The calcium analyzer 500 comprises a peak detection portion 510 arranged to identify calcium peaks in the calcium signal 110 e.g. as described in the foregoing in context of block 320 of the method 300. The calcium analyzer 500 further comprises a peak characteristic derivation portion 520 arranged to derive the respective values of the plurality of peak characteristic for the identified peaks of the calcium signal 110 e.g. as described in the foregoing in context of block 330 of the method 300. The calcium analyzer 500 further comprises a classifier portion 530 arranged to classify each identified calcium peaks into one of the plurality of classes on basis of the peak characteristic values derived for the respective peak and to classify the underlying cardiomyocyte into one of these classes in accordance with these classifications, e.g. as described in the foregoing in context of blocks 350 and 360 of the method 300. The classifier portion 530 may be further arranged to carry out the pre-classification of the calcium signal 110 into one of a normal signal or an abnormal signal, e.g. as described in the foregoing in context of block 340 of the method 300.

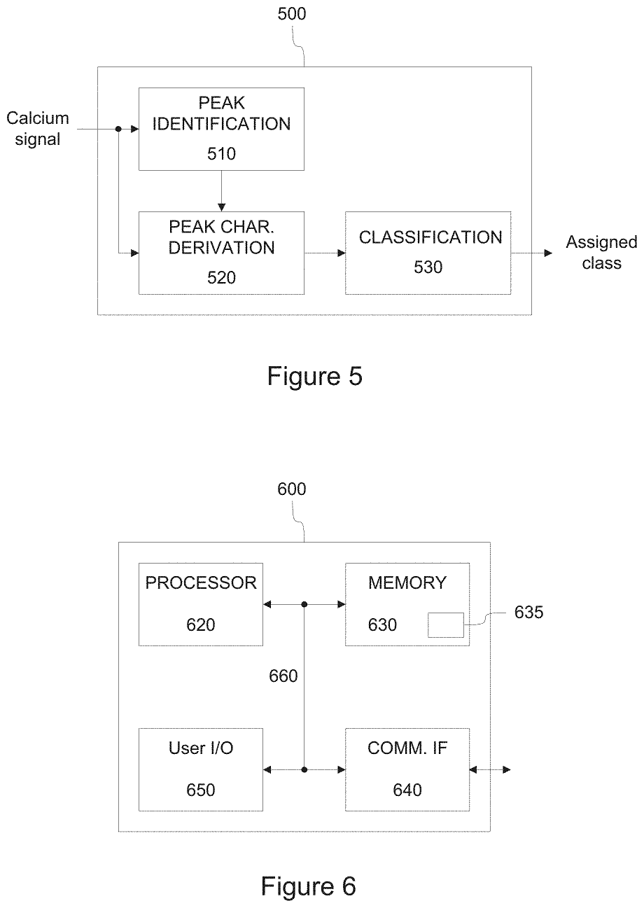

FIG. 6 illustrates a block diagram that depicts some elements of a computing apparatus 600 that may be arranged to implement calcium analysis according to the method 300. The computing apparatus 600 comprises a processor 620, which may serve as a central processing unit (CPU) of the computing apparatus 600. The computing apparatus 600 further comprises a memory 630 for storing data and/or program code for one or more computer programs. The computing apparatus 600 may further comprise a communication interface 640, e.g. a network adapter, which enables wireless and/or wired communication with other devices. The computing apparatus 600 further comprises one or more input/output (I/O) components 650 to enable the computing apparatus 600 to receive input from a user and/or to provide output to the user. The I/O components 650 may include, for example, one or more of the following: a display, a touchscreen, a touchpad, a keyboard or a keypad, a mouse or a pointing device of other type, etc. The computing apparatus 600 may be, for example, a personal computer such as a laptop computer or a desktop computer, a tablet computer, a personal digital assistant (PDA), a mobile phone or a smartphone, etc.

Although in FIG. 6 the processor 620 is depicted as a single component, the processor 620 may be implemented as one or more separate components. Similarly, although the memory 630 is illustrated as a single component, the memory 630 may be implemented as one or more separate components. Some or all of such memory components may be integrated/removable and/or may provide permanent/semi-permanent/dynamic/cached storage.

A portion of the program code stored in the memory 630, when executed by the processor 620, may be arranged to provide an operating system arranged to control operation of the computing apparatus 600. Another portion of the program code stored in the memory 630 may be arranged, when executed by the processor 620, together with the operating system to provide a user interface that allows the user to operate the computing apparatus 600 with the aid of the I/O components 650. Hence, the processor 620 may be arranged to control operation of the computing apparatus 600 in accordance with program code stored in the memory 630 and/or in accordance with user input received via the user interface.

The memory 630 may be further arranged to store a computer program code 635 comprising one or more sequences of one or more instructions that, when executed by the processor 620, causes the computing apparatus 600 to implement at least some of operations, procedures, functions and/or methods described in the foregoing in context of the method 300. The computer program code 635 may constitute that a stand-alone computer program that is executable by the computing apparatus 600 independently of further applications in the framework provided by the operating system. As another example, the computer program code 635 may comprise instructions that are executable in context of another application provided in framework provided by the operating system. An example of such another application is a browser application.

The components of the computing apparatus 600, e.g. the processor 620, the memory 630, the communication interface 640 and the I/O components 650, are typically interconnected by a bus 660. The bus 660 is arranged to provide electrical connection(s) between components of the computing apparatus 600 for transfer of control information, address information and/or data. The computing apparatus 600 serves as an illustrative and non-limiting example of an apparatus that is suitable for executing the program code 635 arranged to implement at least some of operations, procedures, functions and/or methods described in the foregoing in context of the method 300. Hence, an apparatus comprising additional components and/or an apparatus not comprising all components described in context of the computing apparatus 600 may be employed instead.

Features described in the preceding description may be used in combinations other than the combinations explicitly described. Although functions have been described with reference to certain features, those functions may be performable by other features whether described or not. Although features have been described with reference to certain embodiments, those features may also be present in other embodiments whether described or not.

1. A method for calcium analysis on basis of a calcium signal that comprises a time series of samples that are descriptive of calcium level in a cardiomyocyte as a function of time, the method comprising
identifying calcium peaks in the calcium signal;
deriving, for each identified calcium peak, respective values for a plurality of peak characteristics that include at least one of the following:
a change in calcium level indicated by the calcium peak,
a rate of change in calcium level indicated by the calcium peak,
a temporal duration of the calcium peak, and
a time difference to an adjacent calcium peak of the calcium signal;
classifying each identified calcium peak into one of a plurality of classes on basis of said values derived for the respective peak in dependence of predefined classification information that represents said plurality of classes, wherein each of said plurality of classes represents a respective predetermined cardiac condition; and
assigning said cardiomyocyte to one of said plurality of classes in accordance with the respective classifications.

2. A method according to claim 1, further comprising outputting an indication of the outcome of said assignation for further analysis by a medical practitioner.

3. A method according to claim 1, wherein the classification information defines a respective reference point in a K-dimensional space for each of said plurality of classes, where each dimension of said K-dimensional space represents one of said plurality of peak characteristics and wherein said classifying comprises
arranging the respective values for the plurality of peak characteristics derived for a peak into a K-dimensional peak vector to define a point in said K-dimensional space, and
classifying said peak into the class whose reference point is closest to said point in view of a predefined distance measure.

4. A method according to claim 1, wherein the classification information defines a respective partition of a K-dimensional space for each of said plurality of classes, where each dimension of said K-dimensional space represents one of said plurality of peak characteristics and wherein said classifying comprises
arranging the respective values for the plurality of peak characteristics derived for a peak into a K-dimensional peak vector to define a point in said K-dimensional space, and
classifying said peak to the class whose partition of the K-dimensional space includes said point.

5. A method according to claim 3, wherein said reference points or said partitions are defined on basis of training data that includes respective values of said plurality of peak characteristics for respective pluralities of calcium peaks in each of said plurality of classes, the training data thereby including a respective plurality of calcium peaks representing each of the predetermined cardiac conditions.

6. A method according to claim 1, wherein said assigning comprises assigning said cardiomyocyte into that one of said plurality of classes that has the highest number of calcium peaks classified therein.

7. A method according to claim 6, wherein said assigning further comprises providing an indication of a ratio between said highest number of calcium peaks and overall number of identified calcium peaks.

8. A method according to claim 1, wherein said classifying comprises using one of the following classification approaches to classify each calcium peak into one of said plurality of classes:
a random forests classifier,
a binary tree least-squares support vector machine, BT-LSSVM, classifier,
a K-nearest neighbor classifier.

9. A method according to claim 1, wherein said peak characteristics for a peak include one or more of the following:
duration of the ascending side of the peak,
duration of the descending side of the peak, and
time difference between the top of the peak and that of the immediately preceding peak.

10. A method according to claim 9, wherein said peak characteristics for a peak further include one or more of the following:
the change in calcium level indicted by the ascending side of the peak,
the change in calcium level indicated by the descending side of the peak,
maximum rate of change in calcium level in the ascending side of the peak,
absolute value of minimum rate of change in calcium level in the descending side of the peak,
maximum change in the rate of change in calcium level in the descending side of the peak,
absolute value of minimum change in the rate of change in calcium level in the descending side of the peak, and
an area defined by the peak.

11. A method according to claim 1, further comprising
pre-classifying, before said classifying, the calcium signal as one of a normal signal or an abnormal signal; and
classifying each identified calcium peak to one of said plurality of classes in dependence of outcome of the pre-classification.

12. A method according to claim 11, wherein said pre-classification comprises
determining each identified calcium peak as one of a normal peak and an abnormal peak; and
pre-classifying the calcium signal as an abnormal signal in response to determining at least a predefined amount of the identified calcium peaks as abnormal peaks and pre-classifying the calcium signal as a normal signal otherwise.

13. A method according to claim 11, wherein said classifying said cardiomyocyte into one of a plurality of classes in dependence of outcome of the pre-classification comprises one or more of the following:
selecting predefined information that represents said plurality of classes in dependence of the outcome of the pre-classification,
adjusting predefined information that represents said plurality of classes in dependence of the outcome of the pre-classification.

14. A method according to claim 1, wherein said plurality of predetermined cardiac conditions include one of the following:
two or more different inheritable cardiac conditions,
a healthy cardiac condition and at least one inheritable cardiac condition.

15. A method according to claim 14, wherein in said inheritable cardiac conditions include one or more of the following:
catecholaminergic polymorphic ventricular tachycardia, CPVT,
long QT syndrome 1, LQT1,
hypertrophic cardiac myopathy, HCM.

16. (canceled)

17. A non-transitory computer readable medium, on which is stored program code configured to perform the method according to claim 1 when run on a computing apparatus.

18. (canceled)

19. An apparatus for calcium analysis on basis of a calcium signal that comprises a time series of samples that are descriptive of calcium level in a cardiomyocyte as a function of time, the apparatus comprising at least one processor and at least one memory including computer program code for one or more programs, the at least one memory and the computer program code configured to, with the at least one processor, cause the apparatus at least to:
identify calcium peaks in the calcium signal;
derive, for each identified calcium peak, respective values for a plurality of peak characteristics that include at least one of the following:
a change in calcium level indicated by the calcium peak,
a rate of change in calcium level indicated by the calcium peak,
a temporal duration of the calcium peak, and
a time difference to an adjacent calcium peak of the calcium signal;
classify each identified calcium peak into one of a plurality of classes on basis of said values derived for the respective peak in dependence of predefined classification information that represents said plurality of classes, wherein each of said plurality of classes represents a respective predetermined cardiac condition; and
assign said cardiomyocyte to one of said plurality of classes in accordance with the respective classifications.

20. A method according to claim 2, wherein the classification information defines a respective reference point in a K-dimensional space for each of said plurality of classes, where each dimension of said K-dimensional space represents one of said plurality of peak characteristics and wherein said classifying comprises
arranging the respective values for the plurality of peak characteristics derived for a peak into a K-dimensional peak vector to define a point in said K-dimensional space, and
classifying said peak into the class whose reference point is closest to said point in view of a predefined distance measure.

21. A method according to claim 2, wherein the classification information defines a respective partition of a K-dimensional space for each of said plurality of classes, where each dimension of said K-dimensional space represents one of said plurality of peak characteristics and wherein said classifying comprises
arranging the respective values for the plurality of peak characteristics derived for a peak into a K-dimensional peak vector to define a point in said K-dimensional space, and
classifying said peak to the class whose partition of the K-dimensional space includes said point.

22. A method according to claim 4, wherein said reference points or said partitions are defined on basis of training data that includes respective values of said plurality of peak characteristics for respective pluralities of calcium peaks in each of said plurality of classes, the training data thereby including a respective plurality of calcium peaks representing each of the predetermined cardiac conditions.
